(19) United States
(12) Patent Application Publication
(10) Pub. No.: US 2026/0174455 A1
(43) Pub. Date: Jun. 25, 2026

(54) MEDICAL INSTRUMENT

(71) Applicant: KARL STORZ SE & Co. KG, Tuttlingen (DE)

(72) Inventors: Lothar MITZLAFF, Tuttlingen (DE); Juri KÖNIG, Tuttlingen (DE); André FICHTNER, Tuttlingen (DE); Fabian SILBERBERGER, Tuttlingen (DE); Steffen SCHÖNTHALER, Tuttlingen (DE)

(73) Assignee: KARL STORZ SE & Co. KG, Tuttlingen (DE)

(21) Appl. No.: 19/421,379

(22) Filed: Dec. 16, 2025

(30) Foreign Application Priority Data
Dec. 19, 2024 (DE) 10 2024 138 988.5

(51) Int. Cl. A61B 17/29

(52) U.S. Cl. CPC A61B 17/29; A61B 2017/2903; A61B 2017/2938; A61B 2017/2939

(57) ABSTRACT

The disclosure relates to a medical instrument comprising: an end effector with a first end effector element and at least one second end effector element cooperating with the first end effector element; a drive unit with a first drive wheel and a second drive wheel; a transmission unit with a rotatable first intermediate element coupled to the first drive wheel in a motion-transmitting manner, and a rotatable second intermediate element coupled to the second drive wheel in a motion-transmitting manner; and an output unit with a first toothing element that is rotationally fixed to the first end effector element and is coupled to the first intermediate element in a motion-transmitting manner, and a second toothing element which is rotationally fixed to the second end effector element and is coupled to the second intermediate element in a motion-transmitting manner.

### CROSS-REFERENCE TO RELATED APPLICATIONS

This application claims priority of German Patent Application No. DE 10 2024 138 988.5 filed on Dec. 19, 2024, the contents of which are incorporated herein.

### TECHNICAL FIELD

The present disclosure relates to a medical instrument.

### BACKGROUND

In order to provide the required flexibility and/or articulation of medical instruments, currently known medical instruments often have a complex and cost-intensive design. A key problem with current solutions is that the integrated traction cables, which are essential for transferring loads to instrument joints, often have to be routed over deflection radii that are too small. These cables are subjected to high mechanical stresses, particularly due to alternating bending, which significantly reduces their service life. The reusability of such instruments is therefore severely limited due to premature material fatigue and/or wear.

To reduce costs, many users resort to single-use instruments. However, these usually offer a limited number of degrees of freedom in the movement of the medical instrument, which significantly restricts the surgical options and precision of a treating physician. A further problem arises with the geometry of the instruments: angled portions can cause lever arms to be positioned outside the cross-section of a tube or shaft of the medical instrument. This not only increases the risk of injury to the patient, but can also significantly impair the field of view of an endoscope.

### SUMMARY

The object of the disclosure is to, but is not limited to, advantageously developing a medical instrument, in particular for use in a medical robot system, primarily with respect to increasing freedom of movement and reliability. Furthermore, it is an object of the present disclosure, inter alia, to ensure frequent reusability of such a medical instrument and therefore reduce operating costs.

This object is achieved according to the disclosure by the features of the independent claims. Developments of the disclosure can be found in the dependent claims.

The disclosure relates to a medical instrument comprising:
an end effector with a first end effector element and at least one second end effector element cooperating with the first end effector element;
a drive unit with a first drive wheel and a second drive wheel;
a transmission unit with a rotatable first intermediate element which is coupled to the first drive wheel in a motion-transmitting manner, and a rotatable second intermediate element which is coupled to the second drive wheel in a motion-transmitting manner; and
an output unit with a first toothing element which is rotationally fixed to the first end effector element and which is coupled to the first intermediate element in a motion-transmitting manner, and a second toothing element which is rotationally fixed to the second end effector element and which is coupled to the second intermediate element in a motion-transmitting manner.

Such a design makes it possible to provide an advantageously further developed medical instrument. In particular, such a medical instrument can have a high degree of freedom of movement and reliability. In addition, costs for medical applications can be reduced, because a medical instrument of this kind can be reused.

A “medical instrument” is to be understood as a medical tool which is preferably designed to grip, manipulate, hold, cut, and/or otherwise interact with an object to be handled. An object to be handled may preferably refer to any organic and/or inorganic structure. In particular, this means anatomical structures of a patient, such as organs and/or tissues, and/or consumables such as threads, staples, films, swabs, tubes, screws, and/or nails.

The medical instrument can be intended for use in surgical procedures and/or invasive operations. The medical instrument can be part of a medical robot system and/or can at least be functionally coupled to such a system. In some embodiments, the medical instrument can be designed as a hand-held medical instrument. In other words, the medical instrument can be controlled manually, semi-automatically, and/or fully automatically.

An “end effector” is to be understood as a component and/or device of the medical instrument which is preferably arranged at a distal end of the medical instrument, or in other words, close to the patient, during use of the medical instrument. The end effector may be designed to establish physical contact with and/or interact with one and/or more objects to be handled. The end effector can be designed differently depending upon the range of requirements and/or task.

The first end effector element can be configured to interact with, cooperate with, and/or mutually influence the second end effector element in order to perform and/or fulfill a specific task. The first and second end effector elements can be designed to be complementary. The first and second end effector elements can form a positive connection. The end effector elements can, for example, be designed as jaw parts. The jaw parts can be designed to perform the actual function of the medical instrument. For example, the jaw parts can be designed as at least a subcomponent of forceps, tweezers, and/or scissors.

According to a further development, the medical instrument can also comprise a shaft. The shaft can be an elongated and/or cylindrical component. Mechanical components and/or cables can be guided securely and/or in an organized manner through the shaft. The shaft can contribute to the stability of the medical instrument and/or protect kinematic structures against external influences.

The first and/or the second drive wheel can be arranged coaxially with a joint axis which corresponds to a bending axis of the shaft. This space-saving design gives the medical instrument a degree of freedom that allows the distal end and/or a distal shaft portion to pivot about the joint axis relative to a proximal shaft portion. The proximal shaft portion can refer to a portion of the shaft of the medical instrument that faces away from the patient during operation. The distal shaft portion can be pivotable and/or bendable about the joint axis by at least 45°, preferably at least 90° and particularly preferably at least 160°.

The first and/or the second drive wheel can be designed as a mechanical component and/or a transmission component which is configured to transmit at least one drive force and/or at least one drive torque to the first and/or the second intermediate element.

The first and/or the second drive wheel can be designed as a gear. The drive wheels can preferably be made of thermally stable materials, such as metals and/or high-performance polymers, such as polyether ketones. Such a design can be robust against wear and/or high temperatures, thereby significantly increasing the service life of the medical instrument, even with sterilization procedures at high temperatures and/or frequent use.

The first end effector element and/or the second end effector element can be pivoted independently of one another about a pivot axis which runs at an angle, preferably orthogonally, to the joint axis. This can mean that the two end effector elements can be pivoted separately and/or without direct influence from a movement of the other effector element about a reference axis referred to as the “pivot axis.” The pivotability of the first and/or the second end effector element allows for an additional degree of freedom of the medical instrument. The additional degree of freedom can improve the freedom of movement of the medical instrument and/or the precision. This can also contribute to better maneuverability, extended reach, and/or more efficient performance of surgical procedures, in particular in difficult-to-access regions such as patient cavities. The alignment and/or control of the distal shaft portion can occur independently of the control and/or alignment of the end effector and/or of the end effector elements.

Alternatively, the joint axis can extend parallel to the pivot axis about which the end effector elements can be pivoted.

The pivoting movement of the end effector elements can occur in predefined angular steps, but is preferably almost continuous and, particularly preferably, continuous. Each of the end effector elements can be pivotable by at least 45°, preferably at least 90° and particularly preferably at least 160°, about the pivot axis.

According to a further development, the first intermediate element can comprise a first worm shaft and a first intermediate gear, and/or the second intermediate element can comprise a second worm shaft and a second intermediate gear. The first intermediate gear can be coupled to the first worm shaft in a motion-transmitting manner, and/or the second intermediate gear can be coupled to the second worm shaft in a motion-transmitting manner. By means of such a design, movements can be transmitted reliably and/or precisely by rolling movements. Furthermore, this allows for a high degree of compactness in the medical instrument. The first and/or the second worm shaft may comprise a helical, coiling, and/or spiral structure and/or thread that is incorporated into a respective shaft portion or a respective shaft. Alternatively, the first and/or the second worm shaft can be attached as a separate component to the corresponding shaft portion, e.g., via a hub connection, in a rotationally fixed manner.

In some embodiments, the first intermediate gear can be formed integrally with the first worm shaft, and/or the second intermediate gear can be formed integrally with the second worm shaft. The first intermediate gear can be formed monolithically with the first worm shaft, and/or the second intermediate gear can be formed monolithically with the second worm shaft. Such a design can, in particular, ensure increased stability and/or durability.

Alternatively, the first intermediate gear and the first worm shaft and/or the second intermediate gear and the second worm shaft can be designed as separate components. This design can offer advantages in manufacturing, maintenance, and/or modularity, since, for example, damaged parts can be replaced more easily, and/or the design can be adapted more flexibly. Depending upon the requirements for the function, cost, and/or ease of maintenance of the medical instrument, the design of the intermediate elements can vary.

According to an alternative embodiment, the first intermediate element may comprise a first helical gear instead of the first worm shaft and the first intermediate gear, and/or a second helical gear instead of the second worm shaft and the second intermediate gear. The first and/or the second helical gear may preferably have tooth flanks that are at an angle, preferably 45 degrees, to the radial plane. This can increase the reliability and/or durability of the medical instrument.

The first intermediate element can be rotatable about a first rotary axis, and the second intermediate element can be rotatable about a second rotary axis. The intermediate elements can be arranged such that the first rotary axis and the second rotary axis run parallel to one another. The first and/or the second rotary axis can be arranged at an angle, preferably orthogonally, or parallel to the pivot axis. The first and/or the second rotary axis can be arranged at an angle, preferably orthogonally, or parallel to the joint axis.

In some embodiments, the drive unit may further comprise a first traction device which is configured to actuate the first drive wheel and a second traction device which is configured to actuate the second drive wheel. The first and/or the second traction device can set the first and/or the second drive wheel into controlled rotation in both directions. The use of multiple drive units can ensure independent control of the individual drive wheels, resulting in increased precision and flexibility in motion execution. Each drive wheel can therefore be individually controlled, so that complex motion sequences as well as synchronous and/or asynchronous movements can be implemented precisely.

The first traction device can extend parallel to the second traction device, at least partially and preferably for the most part.

The first and/or the second traction device may comprise a cable drive, a tape drive, a belt drive, a chain drive, and/or other transmissions that would appear advantageous to a person skilled in the art, such as rack-and-pinion gears. By means of such a design, movements can be transmitted reliably and/or precisely over a distance. Such a design can be cost-effective to manufacture and/or robust against wear, thereby significantly increasing the service life of the medical instrument despite frequent use, and improving cost-effectiveness.

In some embodiments, the movements of the drive wheels, intermediate elements, and/or end effector elements can be related via linear functions, and these can be superimposed by addition in the case of combined movements. In this way, simple, precise, and/or efficient control of the medical instrument can be achieved, resulting in overall higher system reliability.

According to some embodiments, the medical instrument may comprise a quick-release mechanism which, in a locked state, is configured to secure the end effector elements against axial and/or radial displacement relative to the pivot axis and, in a released state, is configured to allow axial and/or radial displacement of the end effector elements relative to the pivot axis. The quick-release mechanism can be particularly advantageous for medical instruments whose end effector elements wear out very quickly, such as cutting blades in cutting instruments. Such a quick-release mechanism can facilitate the replacement of the end effector elements, since the quick-release mechanism can be opened quickly and preferably without tools by a simple mechanism, such as a lever, button, and/or rotary lock. This can increase user-friendliness and safety, since the replacement can be made without directly touching the end effector elements.

The devices according to the disclosure are not to be limited to the application and embodiment described above. In particular, they can have a number of individual elements, components, and units which differ from a number mentioned herein, in order to fulfill a function described herein. In addition, for the ranges of values specified in this disclosure, values within the stated limits shall also be deemed to be disclosed and to be usable in any manner.

The present disclosure is described below by way of example with reference to the accompanying figures. The drawings, the description, and the claims contain numerous features in combination. A person skilled in the art will also, expediently, consider the features individually and use them in combination as appropriate in the context of the claims.

If there is more than one example of a particular object, only one of them may be provided with a reference sign in the figures and in the description. The description of this example can be transferred accordingly to the other examples of the object. If objects are named using number words, such as first, second, third object, etc., these are used to name and/or assign objects. Accordingly, for example, a first object and a third object may be included, but not a second object. However, a number and/or sequence of objects could also be derived using numerical words.

### BRIEF DESCRIPTION OF DRAWINGS

FIG. 1 is a perspectival view of a medical instrument which has a kinematic structure,

FIG. 2 is a detail view of the kinematic structure of the medical instrument in a first position,

FIG. 3 is a further detail view of the kinematic structure of the medical instrument in the first position,

FIG. 4 is a detail view of the kinematic structure of the medical instrument in a second position,

FIG. 5 is a detail view of one half of the kinematic structure of the medical instrument,

FIG. 6 is a perspectival view of an intermediate element of the medical instrument, and

FIG. 7 is a detail view of a kinematic structure of a medical instrument according to a further embodiment.

### DETAILED DESCRIPTION OF PREFERRED EMBODIMENTS

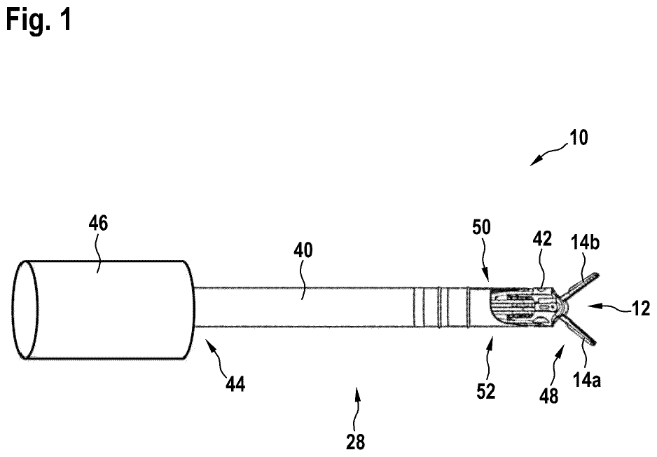

FIG. 1 shows a medical instrument 10 with a shaft 28. The shaft 28 has a proximal shaft portion 40 and a distal shaft portion 42. An interface 46 of the medical instrument 10 is arranged at a proximal end 44 of the proximal shaft portion 40. The interface 46 can be designed to be coupled in a functional and/or controllable manner to a robot device (not shown herein) and/or to a handle. The shaft can be rotated about a rotation axis R. The distal shaft portion 42 of the medical instrument 10, which has an end effector 12, is arranged at a distal end 50 of the proximal shaft portion 40. The end effector 12 comprises a gripping arrangement 48 which has two end effector elements 14a, 14b. The gripping arrangement 48 is shown in an open gripping position.

At this point, it should be noted that the gripping arrangement 48 is to be understood only by way of example and, depending upon the field of application, other end effectors 12 and/or arrangements that appear advantageous to a person skilled in the art may also be provided. A length of the proximal shaft portion 40 can correspond in particular to at least five times, preferably at least ten times and particularly preferably at least fifteen times, the length of the distal shaft portion 42.

The end effector 12, the end effector elements 14a, 14b, and/or the distal shaft portion 42 can be controlled and/or moved by means of a kinematic structure 52 of the medical instrument 10.

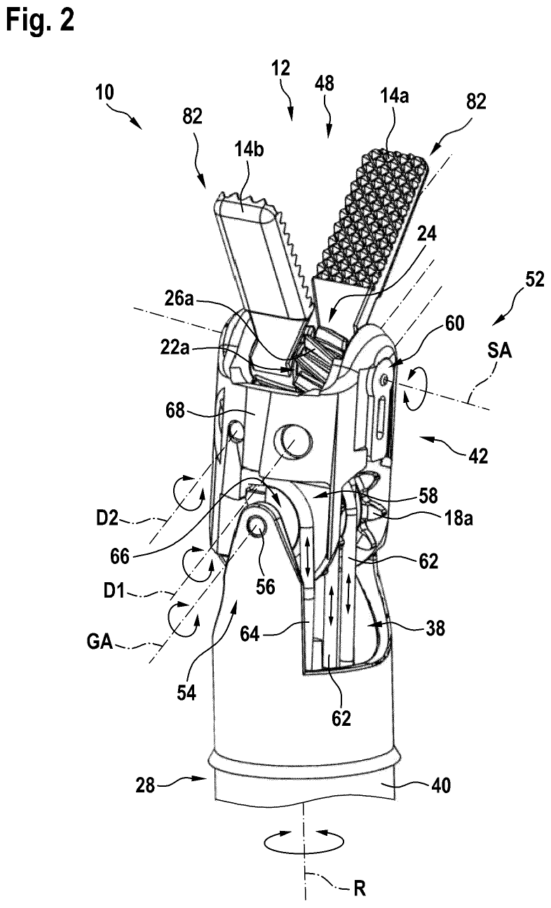

FIG. 2 is a detail view of the kinematic structure 52 of the medical instrument 10 in a first position. The distal shaft end 54 of the proximal shaft portion 40 can be seen, which is captively and movably coupled to a proximal shaft end 58 of the distal shaft portion 42 by means of a first bearing bolt 56. The distal shaft portion 42 is designed such that the longest extent of its cross-section does not extend beyond the cross-section of the proximal shaft portion 40. This can reduce unwanted movements and/or the risk of injury to a patient during treatment by a physician. Furthermore, this design of the medical instrument 10 allows for a less obstructed field of vision for the treating physician.

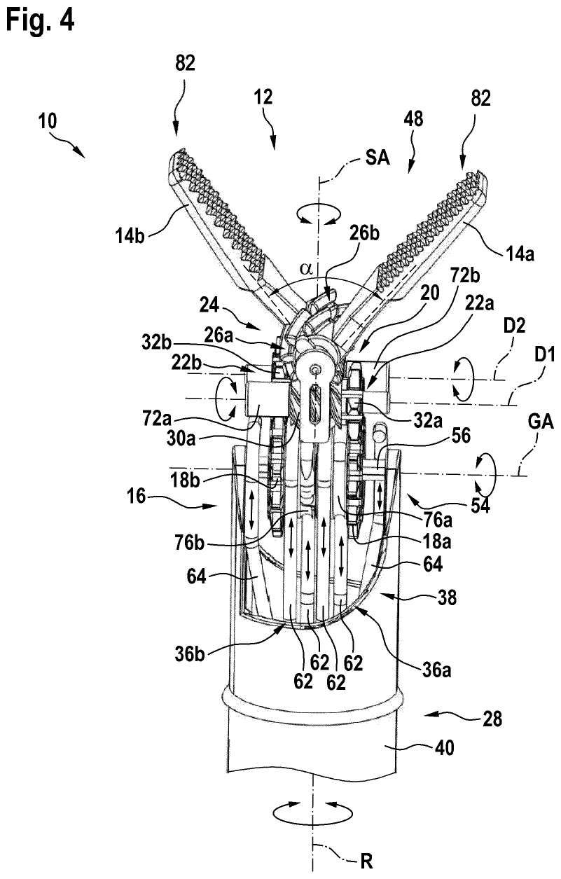

As shown in FIG. 1, the bearing bolt 56 is pivotably and/or fixedly mounted in a wall 68 of the distal shaft portion 42. The distal shaft portion 42 has two coupling portions 66, at which it is articulatedly coupled to the proximal shaft portion 40 via the bearing bolt 56. In the present FIG. 1, only one coupling portion 66 is shown. The other coupling portion 66 is located on the opposite side of the distal shaft portion 42, i.e., on the side that lies on the rear side of the plane of FIG. 1. The coupling portions 66 are each configured to be coupled to a pivot cable 64 of a cable drive 38 in a motion-and/or force-transmitting manner. For a better view of the cable drive, reference can be made to FIG. 4. Depending upon which of the pivot cables 64 is actuated and/or subjected to tension, the distal shaft portion 42 pivots in one direction or the other about a joint axis GA, which preferably runs coaxially with a longitudinal axis of the first bearing bolt 56. If one of the two pivot cables 64 is subjected to tensile stress, the stressed pivot cable 64 is wound up from the coupling portion 66, and the other pivot cable 64 is wound up on the other coupling portion 66. The coupling portions 66 and/or the pivot cables 64 can be interdependently coupled. This means that the coupling portions and/or the pivot cables 64 influence one another, in particular with regard to their winding angle. The winding angle and/or the behavior of each pivot cable 64 may depend upon the winding angle and/or the behavior of each pivot cable 64.

A drive unit 16 with two drive wheels 18a, 18b is arranged on the bearing bolt 56. The drive wheels 18a, 18b are coupled to drive cables 62 via a respective pulley 76a, 76b in a motion-transmitting manner. Both the drive wheels 18a, 18b and the pulleys are arranged coaxially on the first bearing bolt 56. The pulleys 76a, 76b are each coupled to two drive cables 62. Depending upon which pulley 62 is actuated, the first drive wheel 18a and/or the second drive wheel 18b are/is set into a rotary motion about the first bearing bolt 56 and/or the joint axis GA. Depending upon which of the two drive cables 62 coupled to one of the pulleys is actuated, the corresponding drive wheel 18a, 18b is rotated in one direction or the other about the first bearing bolt 56 and/or the joint axis GA. The pulleys 62 and/or the drive wheels 18a, 18b are arranged at least partially within the distal shaft portion 42. The first drive wheel 18a can be rotated independently of the second drive wheel 18b about the joint axis GA and/or the bearing bolt 56 when a corresponding drive cable 62 is actuated.

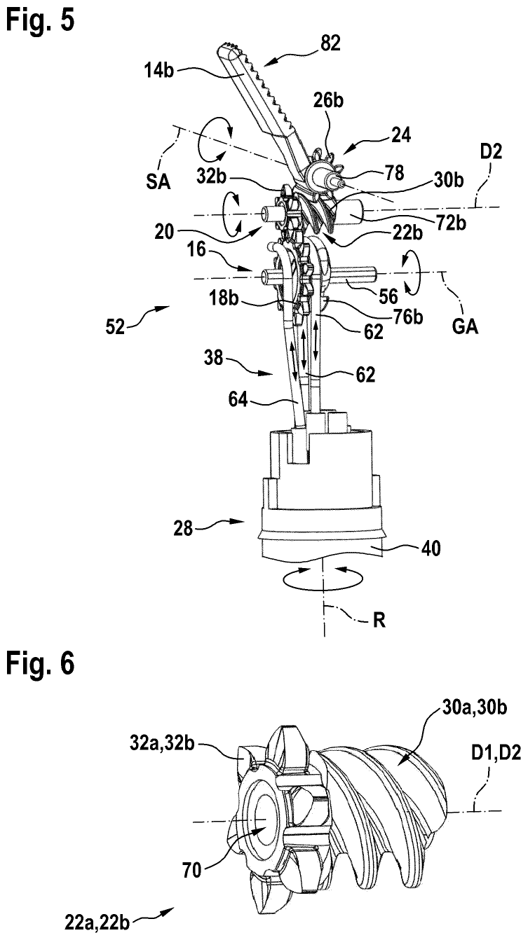

The drive wheels 18a and 18b are coupled to the end effector 12 via a transmission unit 20 in a motion-transmitting manner. The end effector 12 is arranged at a distal region of the distal shaft portion 42 and has the two end effector elements 14a, 14b. In the embodiment shown herein, the two end effector elements 14a, 14b are each configured as a jaw part 82. The two end effector elements 14a, 14b are designed to be complementary, so that they can cooperate with the other end effector element 14a, 14b. The two end effector elements 14a, 14b can be pivoted about a pivot axis SA independently of one another. In the position shown in FIGS. 1 to 5, the two end effector elements 14a, 14b are pivoted relative to one another about the pivot axis SA at an angle α.

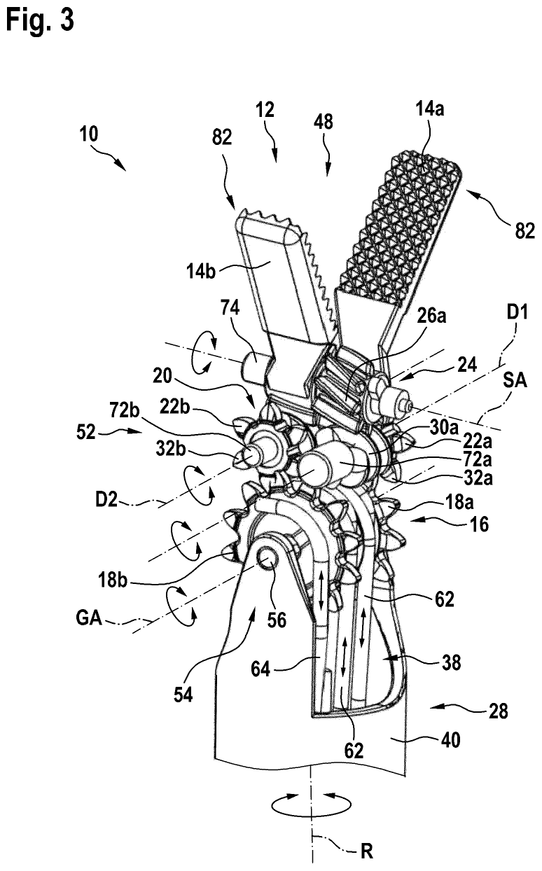

For further details of the kinematic structure 52, reference can be made to FIGS. 3 to 6. In these figures, the wall 68 is hidden. The transmission unit 20, which is designed to transmit movements and/or forces from the drive wheels 18a, 18b to the output unit 24 and/or the end effector elements 14a, 14b, is shown. The transmission unit 20 comprises two intermediate elements 22a, 22b, which, in the embodiment shown herein, comprise a first and a second worm shaft 30a, 30b. The first intermediate element 22a can be rotated about a rotary axis D1, and the second intermediate element can be rotated about a rotary axis D2. The worm shafts 30a, 30b are designed as hollow shafts in this case. Each of the two worm shafts 30a, 30b has an intermediate gear 32a, 32b on one end face. Such an intermediate element 22a, 22b is shown in isolation in FIG. 6. The intermediate gear 32a, 32b is formed integrally and/or monolithically with the worm shaft 30a, 30b. Furthermore, each of the intermediate elements 22a, 22b has an opening 70. Through this opening 70, the intermediate elements 22a, 22b can each be guided onto a stop bolt 72a, 72b of the transmission unit 20. Each of the stop bolts 72a, 72b, like the first bearing bolt 78, is mounted in the wall 68. The stop bolts 72a, 72b each have a stop that prevents displacement of the intermediate elements 22a, 22b in the axial direction and/or along the stop bolts 72a, 72b.

The stop bolts 72a, 72b and/or the intermediate elements 22a, 22b are preferably arranged parallel to the joint axis GA and/or the first bearing bolt 56. The stop bolts 72a, 72b and the intermediate elements 22a, 22b are arranged above the bearing bolt 56. Furthermore, the first intermediate element 22a and the second intermediate element 22b are mirror images of one another and arranged one behind the other. This type of construction makes it possible to use the installation space particularly efficiently and to design the medical instrument 10 to be compact.

The first intermediate element 22a is coupled to the first drive wheel 18a via the first intermediate gear 32a in a motion-transmitting manner. The second intermediate element 22b is coupled to the second drive wheel 18b via the second intermediate gear 32b in a motion-transmitting manner. For motion-transmitting coupling, a toothing of the first intermediate gear 32a engages with a toothing of the first drive wheel 18a, and a toothing of the second intermediate gear 32b engages with a toothing of the second drive wheel 18b. The toothings of the intermediate gears 32a, 32b and those of the drive wheels 18b are complementary.

At the same time, the transmission unit 20 is also coupled to the output unit 24. A first toothing element 26a and/or at least a part of its toothing engage(s), at least partially, in a worm thread of the first worm shaft 30a. Similarly, a second toothing element 26b and/or at least a part of its toothing engage(s), at least partially, in a worm thread of the second worm shaft 30b. In the embodiment shown herein, the toothing elements 26a, 26b are formed integrally with the respective end effector element 14a, 14b. The end effector elements are arranged with their respective end portions on a stop pin 74 or a second bearing bolt 78. The second bearing bolt 78 and/or the stop pin 74 are/is arranged orthogonally to the joint axis GA and the rotary axes D1 and D2.

The joint axis GA, the pivot axis SA, the rotation axis R, and the rotary axes D1 and D2 are indicated by dashed lines in the figures. Degrees of freedom of the drive wheels 18a, 18b, the intermediate elements 22a, 22b, the end effector elements 14a, 14b, and/or the shaft 28 attained by the preceding axes are indicated as arrows. Each of the medical instruments shown can have six degrees of freedom.

To ensure flexible use and high reusability of the medical instrument 10, it features a quick-release mechanism 20. This quick-release mechanism may, for example, comprise a mechanical tensioning and/or clamping mechanism. This mechanism can be opened, for example, by actuating a lever, pressing a button, and/or turning a latch, thereby releasing the end effector elements 14a, 14b. To secure them, the new end effector elements 14a, 14b are slid onto the designated second bearing bolt 78 or stop bolt 72a, 72b, and the quick-release mechanism 60 can securely lock the end effector elements 14a, 14b in place—for example, by friction, pressure, and/or a positive fit.

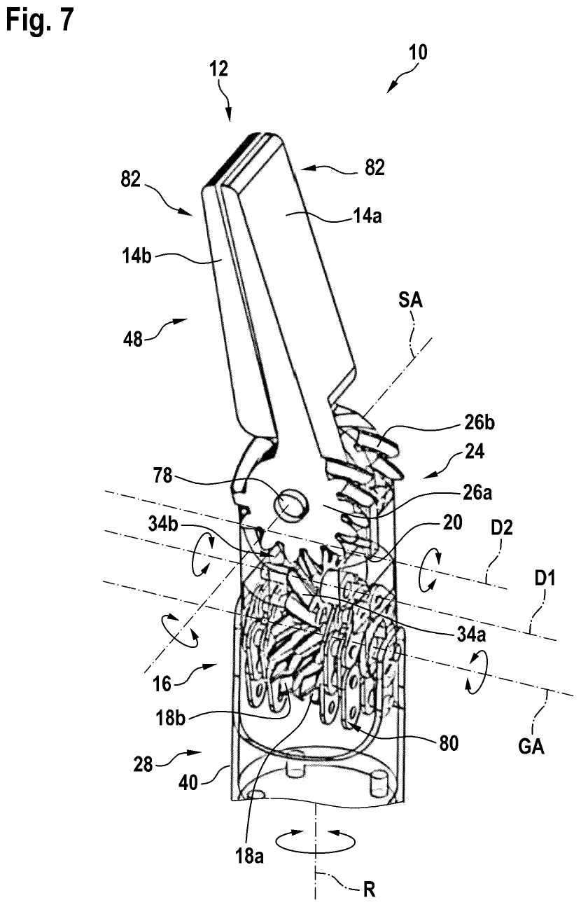

FIG. 7 shows a further embodiment of a medical instrument. The embodiment shown in FIG. 7 differs from that shown in FIGS. 1 to 5 in that, in FIG. 7, the cable drives 38 are replaced by chain drives 80. Furthermore, the intermediate elements 22a, 22b are not designed as worm shafts 30a, 30b and intermediate gears 32a, 32b, but as helical gears 34a, 34b. Furthermore, the gripping arrangement 48 is shown in a closed gripping position, in contrast to FIGS. 1 to 5.

### LIST OF REFERENCE SIGNS

10 medical instrument
12 end effector
14a,b end effector element
16 drive unit
18a,b drive wheel
20 transmission unit
22a,b intermediate element
24 output unit
26a,b toothing element
28 shaft
30a,b worm shaft
32a,b intermediate gear
34a,b helical gear
36a,b traction device
38 cable drive
40 proximal shaft portion
42 distal shaft portion
44 proximal end
46 interface
48 gripping arrangement
50 distal end
52 kinematic structure
54 distal shaft end
56 first bearing bolt
58 proximal shaft end
60 quick-release mechanism
62 drive cable
64 pivot cable
66 coupling portion
68 wall
70 opening
72a,b stop bolt
74 stop pin
76a,b pulley
78 second bearing bolt
80 chain drive
82 jaw part
α angle
GA joint axis
R rotation axis
SA pivot axis
D1 first rotary axis
D2 second rotary axis

1. A medical instrument comprising:
an end effector with a first end effector element and at least one second end effector element cooperating with the first end effector element;
a drive unit with a first drive wheel and a second drive wheel;
a transmission unit with a rotatable first intermediate element which is coupled to the first drive wheel in a motion-transmitting manner, and a rotatable second intermediate element which is coupled to the second drive wheel in a motion-transmitting manner; and
an output unit with a first toothing element which is rotationally fixed to the first end effector element and which is coupled to the first intermediate element in a motion-transmitting manner, and a second toothing element which is rotationally fixed to the second end effector element and which is coupled to the second intermediate element in a motion-transmitting manner.

2. The medical instrument according to claim 1, further comprising:
a shaft, wherein the first and/or the second drive wheel are/is arranged coaxially with a joint axis which corresponds to a bending axis of the shaft.

3. The medical instrument according to claim 2, wherein the first end effector element and/or the second end effector element can be pivoted independently of one another about a pivot axis which runs orthogonally to the joint axis.

4. The medical instrument according to claim 1, wherein the first intermediate element comprises a first worm shaft and a first intermediate gear, and/or the second intermediate element comprises a second worm shaft and a second intermediate gear, wherein the first intermediate gear is coupled to the first worm shaft in a motion-transmitting manner, and/or the second intermediate gear is coupled to the second worm shaft in a motion-transmitting manner.

5. The medical instrument according to claim 4, wherein the first intermediate gear is formed integrally with the first worm shaft, and/or the second intermediate gear is formed integrally with the second worm shaft.

6. The medical instrument according to claim 1, wherein the first intermediate element comprises a first helical gear, and/or the second intermediate element comprises a second helical gear.

7. The medical instrument according to claim 1, wherein the drive unit further comprises a first traction device which is configured to actuate the first drive wheel and a second traction device which is configured to actuate the second drive wheel.

8. The medical instrument according to claim 7, wherein the first traction device and/or the second traction device comprise(s) a cable drive, a tape drive, a belt drive, and/or a chain drive.

9. The medical instrument according to claim 1, wherein movements of the drive wheels, the intermediate elements, and/or the end effector elements are related via linear functions, and can be superimposed by addition in the case of combined movements.

10. The medical instrument according to claim 3, further comprising:
a quick-release mechanism which, in a locked state, is configured to secure the end effector elements against axial and/or radial displacement relative to the pivot axis and which, in a released state, is configured to allow axial and/or radial displacement of the end effector elements relative to the pivot axis.
